(19) United States
(12) Patent Application Publication
(10) Pub. No.: US 2005/0025814 A1
(43) Pub. Date: Feb. 3, 2005

(54) Absorbing article for skin care and antibiotic and manufacture method thereof

(76) Inventor: Shih-Chiang Lin, Paipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/923,803

(22) Filed: Aug. 24, 2004

Related U.S. Application Data
(62) Division of application No. 10/271,741, filed on Oct. 17, 2002.
Application No. 10/271,741 is a continuation-in-part of application No. 09/785,281, filed on Feb. 20, 2001, now abandoned.

(51) Int. Cl. A61K 9/70

(52) U.S. Cl. 424/443

(57) ABSTRACT

An absorbing article for skin care and antisepsis has a substrate having absorbing ability for liquid and powder, and an augmentation layer, which is a uniform thin layer composed by a chitosan, and said augmentation layer is coated on the substrate, wherein said augmentation layer is not dissolved by water, rendering no mud and wet. The augmentation layer has natural biological care to skin, and has deodorization, antibiotic and sterilization effect, thus preventing bedsore.

### FIELD OF THE INVENTION

The present invention relates to an absorbing article for skin care and antisepsis, especially to a substrate of the absorbing article coated with chitosan layer, wherein the substrate can be used as absorbing article for protecting skin.

### BACKGROUND OF THE INVENTION

The conventional diapers or sanitary napkins are made of unwoven cloth, which are stifling after long time use. The user may have the problem of bedsore, heat rash or tainted and will be very uneasy. To solve this problem the unwoven cloth is coated with natural or artificial materials such as mineral jelly or aloe, or waterless lubricant. However, the surface of the unwoven cloth will be hardened and the friction is increased to result more unease. Moreover, in above treatment for unwoven cloth, the unwoven cloth is treated by chemical and certain harmful material such as P is remained. The residual phosphorus is harmful to human after long-term use. In general, the material for absorbing article puts much emphasis on comfortable aspect for people. In prior art, the chitosan powder is applied to the surfaces of the absorbing article by spraying. When the power is mixed with water, it become muddily. It makes user feel adhesive and wet.

### SUMMARY OF THE INVENTION

It is the object of the present invention to provide an absorbing article for skin care and antibiotic. The material used for the absorbing article should block the natural organism growing on the cloth and be lubricative to the skin. The substrate is mainly composed of cloth with a chitosan augmentation layer. The chitosan is a natural high molecular number chitin abstracted from shell of shrimp and crab or bone of cuttle, and is processed by deacetylation step. The degree of deacetylation is generally 65-99% and, most often, 70-90%. The chitosan augmentation layer forms a protective layer with natural biological care to skin, and has deodorization, antibiotic and sterilization effect, thus preventing bedsore.

The absorbing article for skin care and antibiotic according to the present invention has a substrate having absorbing ability for liquid and powder, and an augmentation layer, which is a uniform thin layer composed by a chitosan, and the augmentation layer is coated on the substrate, wherein said augmentation layer is not dissolved by water, rendering no mud and wet feeling to user.

The various objects and advantages of the present invention will be more readily understood from the following detailed description when read in conjunction with the appended drawing, in which:

### BRIEF DESCRIPTION OF DRAWINGS

FIG. 1 is the perspective view of the present invention;

FIG. 2 is the exploded view of the present invention;

FIG. 3 is a perspective view showing the manufacture of the present invention;

FIG. 4 showing the chitosan is applied to a plurality layer for the substrate; and

FIG. 5 shows another preferred embodiment of the present invention.

### DETAILED DESCRIPTION OF THE INVENTION

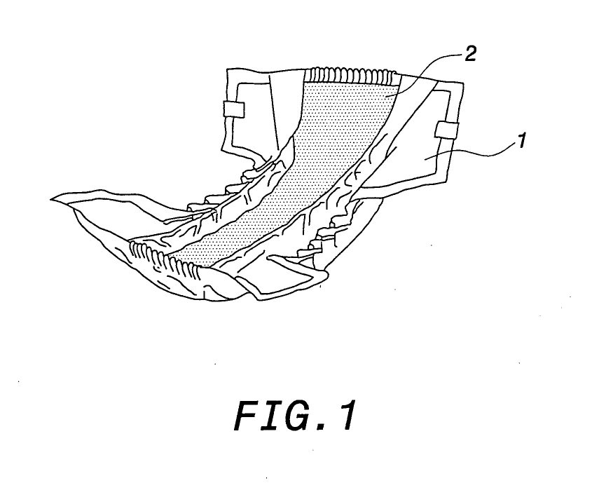

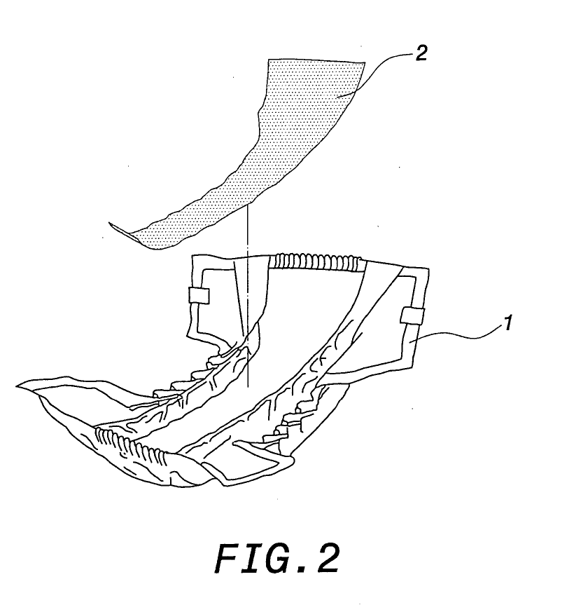

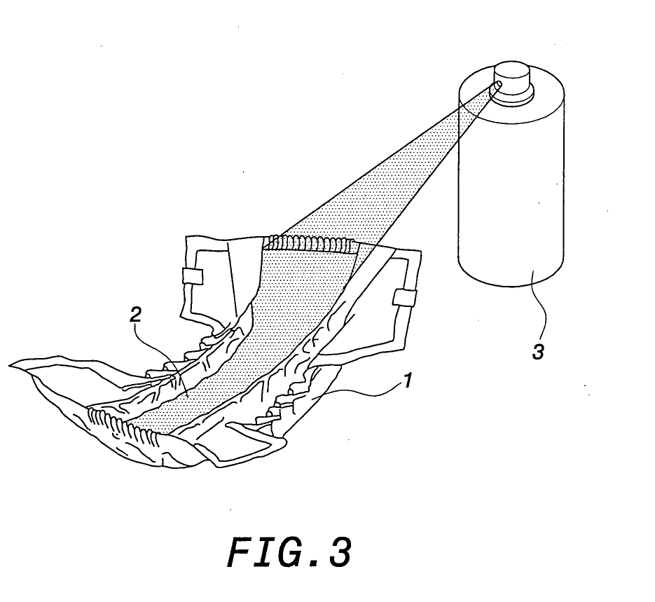

With reference now to FIGS. 1 to 4, the present invention provides an absorbing article for skin care and antibiotic, which comprises a substrate 1 and an augmentation layer 2. The substrate is an absorbing body, which can absorb liquid or powder. In this case, the absorbing body absorbs liquid such as urine or menses secretion. For example, the absorbing body can be sanitary napkin or nappy, which can absorb large amount of liquid or powder on the surface thereof. In the case that the powder is egestion or dirt, the absorbing body can be nursing pad or pet pad.

The manufacture method of absorbing article in first embodiment of the present invention comprised the steps: 1) preparing chitosan solution is prepared, a chitosan dissolved in a 1% ˜10% neutral or acid solution. The neutral or acid solution includes citric acid formic acid acetic acid N-acetylglycine acetylsalicylic acid furmaric acid glycolic acid iminodiacetic acid itaconic acid HCl 2-pyrrolidone-5-carboylic acid salicylic acid succinamic acid succinic acid ascorbic acid aspartic acid glutaric acid malonic acid pyruvic acid and sulfonyl diacetic acid. The neutral or acid solution has pH value of 17.5. The concentration of the chitosan solution is 0.05˜5% (w/w); 2) filling a sprayer having corkscrew nozzle with the chitosan solution and sprinkling the chitosan solution (1˜100 gsm) for coating a uniform thin layer on a substrate. The uniform thin layer is an augmentation layer, wherein the substrate is tissue paper non woven acquisition layer airlaid fluff pad PE film or PP film. The augmentation layer is not dissolved to make people feel mud and wet.

In the second embodiment of the present invention, the step 2) further comprises the steps listing below: After coating a layer of the chitosan solution, drying it via infrared rays or heat wind or natural wind.

In the third embodiment of the present invention, instead of the step 2) sprinkling the chitosan solution by soaking the substrate to the chitosan solution directly.

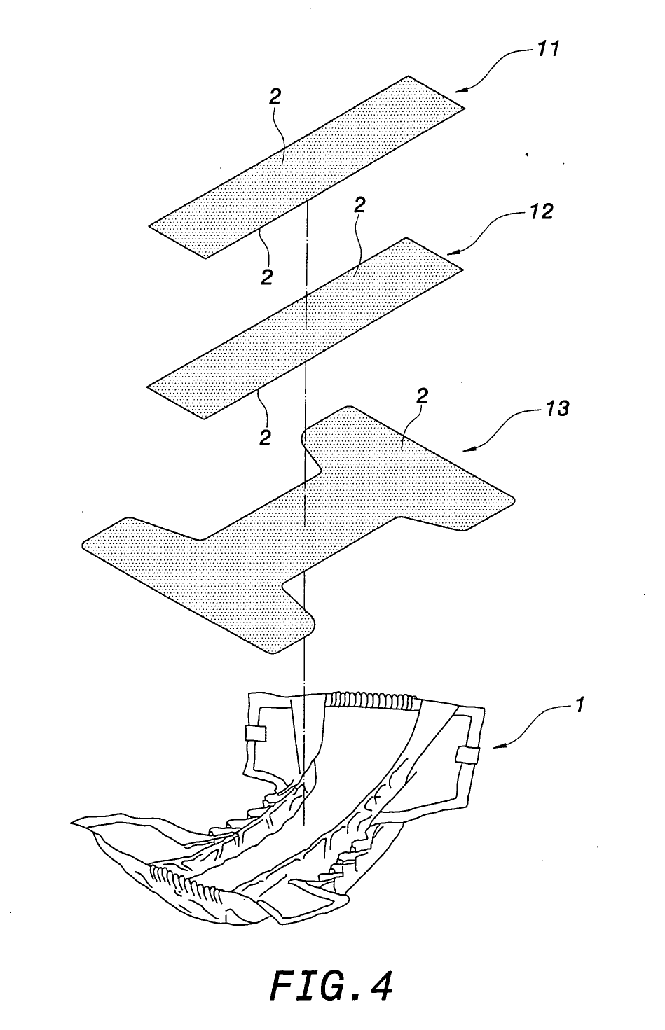

FIG. 4 shows a preferred embodiment of the present invention wherein the substrate 1 is a nappy and only the portion of the substrate 1 applied with augmentation layer is demonstrated. The portion of the substrate 1 applied with augmentation layer comprises an upper cloth layer 11, a guiding and absorbing layer 12 and a kernel absorbing layer 13. The upper cloth layer 11 and the guiding and absorbing layer 12 are applied with chitosan on the outer and inner surfaces thereof, while the kernel absorbing layer 13 is applied with chitosan on the outer surfaces thereof only. In this preferred embodiment, the kernel absorbing layer 13 comprises a cotton layer on outer side and a reinforcing cotton layer on inner side. The guiding and absorbing layer 12 is arranged on outer side of the kernel absorbing layer 13; and the upper cloth layer 11 is arranged on outer side of guiding and absorbing layer 12. The upper cloth layer 11 and the guiding and absorbing layer 12 are made of unwoven cloth. After those layers are arranged on the substrate 1, the chitosan solution is applied to the substrate by a nozzle 3. Afterward, the substrate 1 is naturally dried or dried by a drying process.

The chitosan augmentation layer provides a protective layer on the substrate 1 and cares the skin in a moderate and biological way. The chitosan augmentation layer provides deodorization, antibiotic and sterilization effect and prevents bedsore.

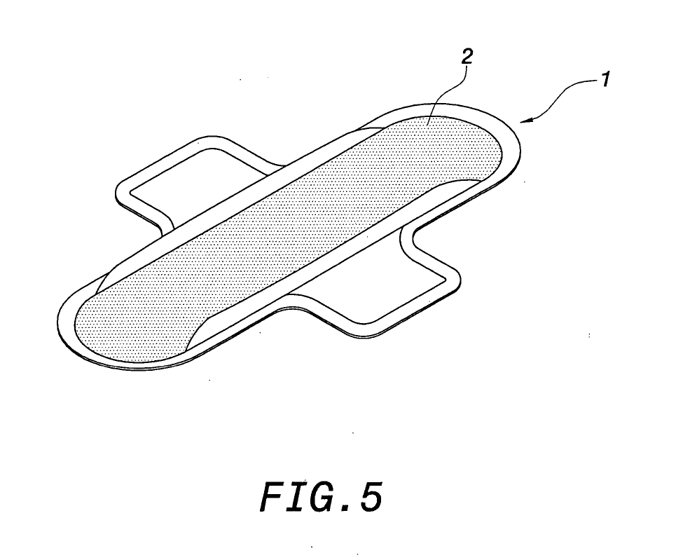

FIG. 5 shows another preferred embodiment of the present invention wherein the substrate 1 is a sanitary napkin.

The added chitosan has excellent stability for long-time storage, and does not harden the cloth. The chitosan is added to the cloth to enhance the comfort of the cloth. The chitosan will have hollow shape after reacting with acid solution to provide isolation for cloth upon combining with cloth, thus isolating the harmful and minute metal content of the cloth.

The antiseptic chitosan can also be absorbed by human and will activate human cell, thus enhancing immunity. The chitosan can also be added to fiber to provide sweat-absorbing, waterproof, antiseptic, deodorization.

To sum up, the protective film formed by spraying chitosan according to the present invention has the advantages of decreasing friction and reducing disease.

Although the present invention has been described with reference to the preferred embodiment thereof, it will be understood that the invention is not limited to the details thereof. Various substitutions and modifications have suggested in the foregoing description, and other will occur to those of ordinary skill in the art. Therefore, all such substitutions and modifications are intended to be embraced within the scope of the invention as defined in the appended claims.

1-2. (canceled).

3. A manufacture method of absorbing article for skin care and antibiotic, comprising the steps:
a) a chitosan solution prepared, a chitosan dissolved in a 1%˜10% neutral or an acid solution, said neutral or acid solution being pH 1˜7.5, and a concentration thereof being 0.05˜5%;
b) The chitosan solution sprinkled to a substrate via a sprayer having corkscrew nozzle, and coating an augmentation layer on the substrate, wherein said augmentation layer is not dissolved by water, rendering no mud and wet.

4. The manufacture method of absorbing article for skin care and antibiotic as in claim 3, wherein said neutral or acid solution includes citric acid or formic acid or acetic acid or N-acetylglycine or acetylsalicylic acid or furmaric acid or glycolic acid or iminodiacetic acid or itaconic acid or HCl or 2-pyrrolidone-5-carboylic acid or salicylic acid or succinamic acid or succinic acid or ascorbic acid or aspartic acid or glutaric acid or malonic acid or pyruvic acid or sulfonyl diacetic acid.

5. The manufacture method of absorbing article for skin care and antibiotic as in claim 3, wherein said substrate is one of a tissue paper, a non woven, a acquisition layer, a airlaid, a fluff pad, a PE film, and PP film.
